(19) United States
(12) Patent Application Publication
(10) Pub. No.: US 2009/0177029 A1
(43) Pub. Date: Jul. 9, 2009

(54) Sexual stimulation device

(75) Inventor: Michelle Alilovich, Libertyville, IL (US)

Correspondence Address: FACTOR & LAKE, LTD, 1327 W. WASHINGTON BLVD., SUITE 5G/H, CHICAGO, IL, 60607, US

(73) Assignee: BIG BANG PRODUCTIONS, LLC, Northbrook, IL (US)

(21) Appl. No.: 12/287,536

(22) Filed: Oct. 10, 2008

Related U.S. Application Data
(60) Provisional application No. 60/978,826, filed on Oct. 10, 2007.

(51) Int. Cl. A61F 5/00; A61H 1/00

(52) U.S. Cl. 600/38; 601/15

(57) ABSTRACT

A sexual stimulation device having an outer skin containing heated, raised ridges surrounding an internal mechanism including two housings, a drive motor, a disc mechanically engaged with the drive motor, a shaft having a planar surface with substantially perpendicular protrusions attached at one end, and angled surfaces attached to the inner surface of at least one of the housings. As the motor and disc rotate, the shaft is translated laterally, pushing the attached planar surface and disc towards the angled surfaces, causing at least one of the housings to expand, thereby increasing the surface area of the surrounding outer skin.

### RELATED APPLICATIONS

The present invention claims priority to U.S. Provisional Patent Application Ser. No. 60/978,826, filed Oct. 10, 2007, entitled Sexual Stimulation Device; and in which its contents are incorporated by reference herein.

### FIELD OF THE INVENTION

The present invention relates to sexual stimulation devices. Specifically, the present invention relates to vibrating sexual stimulation devices with heating and shape changing capabilities.

### BACKGROUND OF THE INVENTION

Numerous devices are commercially available which are adapted to enhance sexual function and to provide sexual stimulation. The majority of the devices that are commercially available may be generally described as either a dildo, i.e. a non-vibratory article adapted for either anal or vaginal insertion, or a vibrator, i.e. an article including means for generating a vibration to enhance sexual stimulation. Although dildos and vibrators come in wide array of designs, styles and sizes, they tend to be in elongated, fixed shapes. While there is a wide variety in the designs, styles, and sizes of commercially available dildos and vibrators, it would be advantageous to have a vibrator that contains an apparatus for increasing or decreasing the circumference of the vibrator to increase comfort and/or enhance stimulation. It would be an additional advantage to create a variable size vibrator that contains a heating element to further enhance stimulation.

### SUMMARY OF THE INVENTION

The present invention is directed to a sexual stimulation device having a flexible outer skin surrounding an inner mechanism. The flexible outer skin may contain raised ridges in various shapes and sizes, which may be in either a random or a predetermined pattern, to enhance stimulation. The raised ridges may be further heated via infrared heating elements or the like to further enhance stimulation.

In one embodiment of the present invention, the sexual stimulation device is provided at one end with a perpendicular protrusion. The perpendicular protrusion is covered with the outer skin and may take any shape. The perpendicular protrusion may further include a vibrating motor enclosed within the area surrounded by the outer skin. The outer skin surrounding the perpendicular protrusion may further include ridges or the like in the surface. As in the body of the device, the ridges contained within the outer skin surrounding the protrusion may be of any shape and size, may be in a random or predetermined pattern, and may include infrared heating elements or the like.

In another embodiment of the present invention, the inner mechanism surrounded by the outer skin comprises two housings made of a rigid material surrounding: a drive motor; a disc mechanically connected to the drive motor; a shaft engaged with an opening in the disc and connecting the two housings; a planar surface connected to the end of the threaded shaft extending longitudinally within the housing; at least one guide pin extending in a perpendicular direction from the planar surface; at least one angled surface extending substantially parallel to the planar surface from the inner surface of the housing; and at least one vibrating motor.

In a further embodiment of the present invention, a means for expanding and contracting at least a portion of the device is included. At least one of the two housings preferably includes at least two separable panels forming a cylindrical shape substantially surrounding the guide pins and the planar surface, which may include inclined or angled surfaces extending from the at least two separable panels inner surface.

In a still further embodiment of the present invention, as the motor operates it rotates the disc which then causes the shaft to translate laterally away from the disc and into the opposite housing. As the shaft laterally translates, the connected planar surface and guide pins translate in the same direction, causing the guide pins to engage the angled surfaces within the second housing. As the guide pins engage the angled surface in the opposing housing, the opposing housing expands, causing the surrounding outer skin to increase in surface area. When the motor is reversed, the shaft, planar surface, and guide pins move in the opposite direction, causing the opposing housing to contract which in turn decreases the surface area of the surrounding outer skin.

In yet another embodiment of the present invention, the inner mechanism surrounded by the outer skin comprises two housings made of a rigid material surrounding: a drive motor; a coiled spring; and, at least one vibrating motor.

These and other aspects and attributes of the present invention will be discussed with reference to the following drawings and accompanying specification and claims.

### BRIEF DESCRIPTION OF THE DRAWINGS

FIG. 1 shows a front side view of one embodiment of the present invention being covered in an outer skin.

FIG. 2 shows an interior portion of the present invention shown in FIG. 1, wherein the inner housing and mechanism is in a fully contracted position.

FIG. 3 shows an interior portion of the present invention shown in FIG. 1, wherein the inner housing and mechanism is in a fully expanded position.

FIG. 4 shows a partial perspective view of a portion of the present invention, wherein the inner housing and mechanism is in the fully contracted position.

FIG. 5 shows a partial perspective view of a portion of the present invention, wherein the inner housing and mechanism is in the fully expanded position.

FIG. 6 shows a partial plan view of the inner housing and mechanism of the present invention shown in FIG. 1 in the fully contracted position.

FIG. 7 shows a partial plan view of the inner housing and mechanism of the present invention shown in FIG. 1 in the fully expanded position.

FIG. 8a shows a longitudinal cross section of the inner housing and mechanism covered by an outer skin taken along line 8-8 in FIG. 7.

FIG. 8b shows a longitudinal cross section of the inner housing and mechanism covered by an outer skin when the present invention shown in FIG. 1 is in its fully contracted position.

FIG. 9 shows an embodiment of the inner housing with a coiled spring contained therein.

### DETAILED DESCRIPTION OF THE PRESENT INVENTION

While the present invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

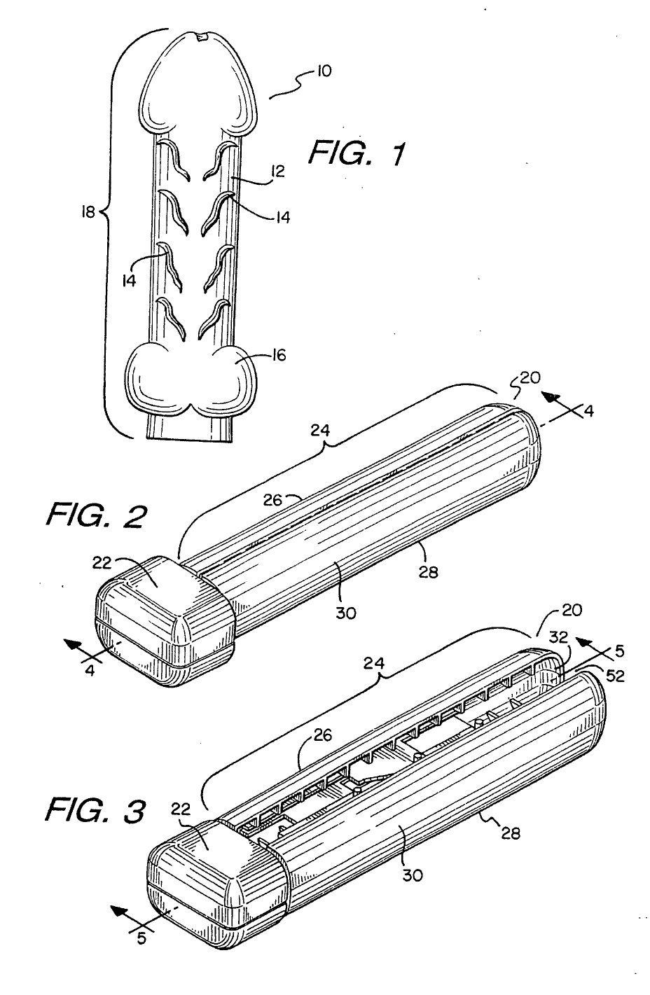

As shown in FIG. 1, the present invention relates to a sexual stimulation device 10 having a main body 18 covered by an outer skin 12. The outer skin 12 may include raised ridges 14 that generally comprise a soft, semi-pliable material that mimics human flesh. Some materials that may be used for the outer skin 12 include, and are not limited to: Silicon, Kraton, or Non-Porus plastic. The raised ridges 14 or skin 12 may be heated in order to enhance stimulation. Heating may occur via infrared heating elements contained within the raised ridges 14 or within the surface of the outer skin 12.

The device 10 may also have attached at one end a semi-spherically shaped protrusion 16 extending in a generally perpendicular direction from the main body 18. The outer skin 12 may cover the circular shaped perpendicular protrusion 16. The circular shaped perpendicular protrusion 16 may have enclosed within a vibrating motor, shown for example in FIG. 2 as a vibrating motor 40.

Contained within the outer skin 12 of the sexual stimulation device 10 is an inner mechanism 20 having various elements for vibrating, expanding, and contracting at least a portion of the device 10 to increase the surface area of outer skin 12. One embodiment of the inner mechanism 20 is shown in FIGS. 2 and 3.

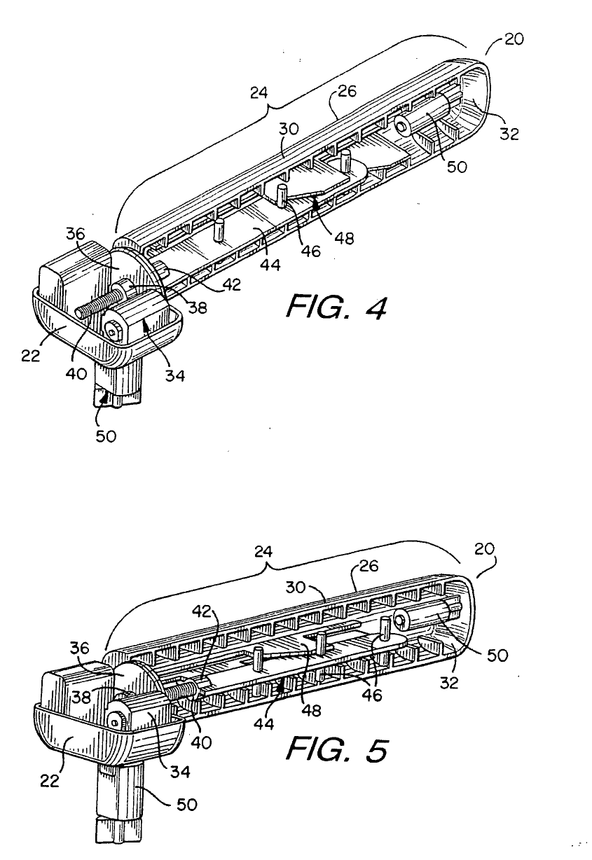

As shown in FIGS. 2 and 3, the inner mechanism 20 is in the fully contracted and fully expanded position, respectively. The inner mechanism 20 may include two or more distinct housings—a first housing 22 taking the form of a rectangular prism, and a second housing 24 taking the form of a generally elongate cylinder—in which a means for expanding and contracting at least a portion of the device 10 may be contained within one or both housings. The cylindrical portion of the second housing 24 may be formed by two or more separable, discrete panels 26, 28, each having an outer surface 30 and an inner surface 32. The outer surface 30 comprises the portion of the inner mechanism 20 that is pushed outwardly to expand the device 10 and increase the surface area of the surrounding outer skin 12. The components for expanding and contracting device 10 contained within the inner mechanism 20 are better seen in FIGS. 4 and 5, which are partial perspective views of FIGS. 2 and 3, respectively.

As shown in FIGS. 4 and 5, contained with the first housing 22 is a drive motor 34 which is mechanically engaged with a disc 36 having an opening 38 there through. Disc 36 may be operatively connected with drive motor 34 in any manner known in the art—for example, using a drive belt or gear teeth. A shaft 40, preferably threaded, is cooperatively engaged with the disc 36 via the opening 38. As the drive motor 34 rotates the disc 36 in a first direction—either clockwise or counterclockwise—the shaft 40 is laterally translated through the opening 38 and extended into the second housing 24. Essentially, the combination of the drive motor 34, disc 36, and shaft 40 may take any embodiment wherein the rotational energy of the drive motor 34 is converted to translation energy to laterally translate shaft 40. When the drive motor 34 is reversed and the disc 36 is rotated in a second direction, the shaft 40 is withdrawn from the second housing 24 and is substantially pulled back into the first housing 22. However, as seen in FIG. 4, when in the fully contracted position, at least a portion of shaft 40 remains within the second housing 24 at all times.

Connected to the shaft 40 at connection point 42 in the second housing 24 is a planar surface 44 having guide pins 46 extending substantially perpendicularly there from. As the drive motor 34 rotates the disc 36, and the shaft 40 laterally translates further into the second housing 24, and the planar surface 44 and the substantially perpendicular guide pins 46 are pushed away from the disc 36 and further into the second housing 24. As the planar surface 44 is pushed further into the second housing 24, the guide pins 46 engage corresponding angled or inclined surfaces 48 attached to inner surfaces 32. As the guide pins 46 are pushed further into the second housing 24, they slide along the angled surfaces 48 and push the separable panels 26, 28 apart, expanding the second housing 24 and thereby increasing the surface area of the surrounding outer skin 12. FIG. 5 shows the inner mechanism 20 in the fully expanded position wherein guide pins 46 are at the innermost portion of the corresponding angled surfaces 48. When the drive motor 34 is reversed, shaft 40 is pulled back out of the second housing 24, causing the guide pins 46 to slide along the corresponding angled surfaces 48 in the direction towards disc 36. Moving the guide pins 46 in this direction causes the second housing 24 to contract, thereby decreasing the surface area of the surrounding outer skin 12.

As seen in FIGS. 4 and 5, the inner mechanism 20 may also contain a vibrating motor 50 to enhance stimulation. Although vibrating motor 50 is shown in FIGS. 4 and 5 to be located proximate one end of the second housing 24, it may be located anywhere within the inner mechanism 20; and it is to be understood by one of ordinary skill in the art that the inner mechanism may contain more than one vibrating motor. In one configuration, a second vibrating motor 50 is located in the semi-spherical shaped, generally perpendicular protrusion 16, which itself extends substantially perpendicular from the first housing 22.

Both the drive motor 34 and vibrating motor 50 may be controlled in any manner known by one of ordinary skill in the art, e.g., wired or wireless remote control via buttons attached to the device, turning a knob, etc.

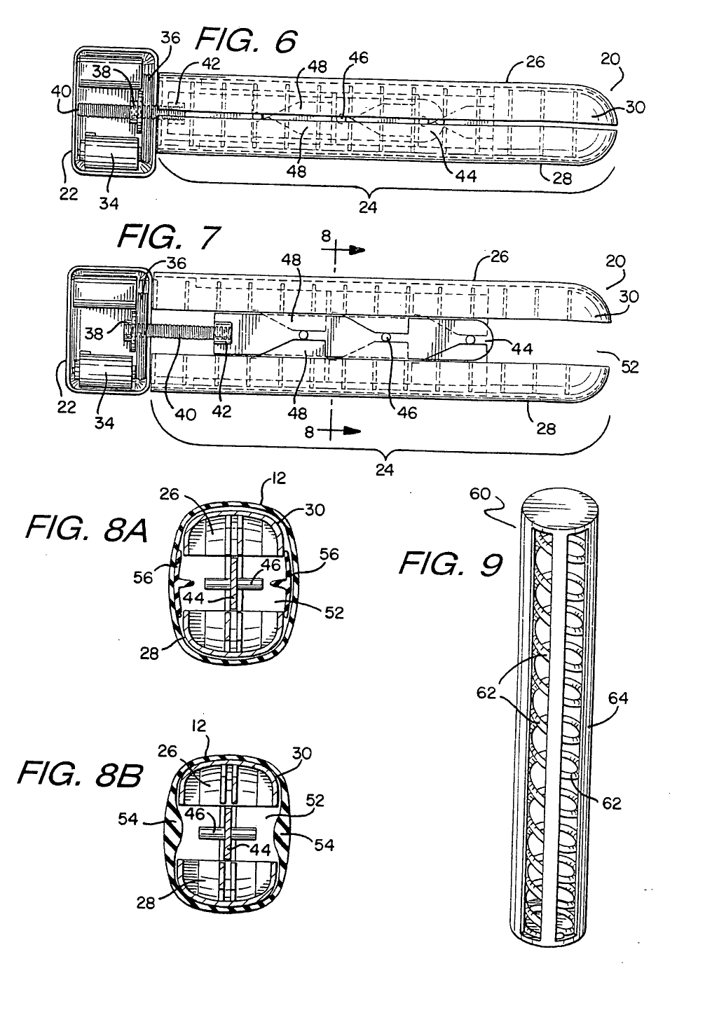

Referring now to FIGS. 6 and 7, a top plan view shows the present invention depicted in FIGS. 2 and 3 in its respective contracted and expanded positions. As seen in FIG. 7, the angled surfaces 48 are located on either side (above or below) of the planar surface 44 with the angled surfaces 48 located “below” the planar surface 44 indicated by dashed lines. Thus, the guide pins 46 extend above and below the planar surface 44 and engage the corresponding angled surface 48, which facilitates the expansion of the second housing 24 from both the top and bottom of planar surface 44.

As seen in FIGS. 3 and 7, when the second housing 24 is in the fully expanded position, a gap 52 forms between the separable panels 26 and 28. As seen in FIG. 8b, in one embodiment, outer skin 12 contains a thicker center portion 54 which expands to cover this gap and provide rigidity when in the fully expanded position. FIG. 8b shows the device 10 and inner mechanism 20 in the fully expanded position with outer skin 12 surrounding the outer surface 30 of the inner mechanism 20. In this position, the thicker center portion 54 of the outer skin 12 expands slightly to cover the gap between panels 26 and 28 while providing enough stiffness to maintain a semi-rigid, cylindrical structure.

FIG. 8a shows an alternative embodiment wherein a flexible joint 56 covers the gap 52 between the separable panels 26 and 28. This flexible joint 56 acts to maintain a semi-rigid, cylindrical shape when the device 10 is in the fully expanded position. Though not shown, it is readily apparent by those of ordinary skill in the art that when the device 10 is in the semi or fully contracted position, the flexible joint 56 lays between outer skin 12 and outer surface 30 of second housing 24. When in the fully contracted position, the panels 26, 28 of the second housing 24 close around the perpendicular portion of flexible panel 56.

FIG. 9 depicts an alternative embodiment for expanding and contracting the device of the present invention wherein the inner housing and mechanism 60 includes panels 64 and a coiled spring 62. In this embodiment, as the coiled spring is stretched, the panels 64 and surrounding outer skin 12 are expanded, thus increasing the surface area of the outer skin.

It is to be understood that additional embodiments of the present invention described herein may be contemplated by one of ordinary skill in the art and the scope of the present invention is not limited to the embodiments disclosed. While specific embodiments of the present invention have been illustrated and described, numerous modifications come to mind without significantly departing from the spirit of the invention, and the scope of protection is only limited by the scope of the accompanying claims.

What is claimed is:

1. A sexual stimulation device comprising:
an inner mechanism having a housing having an inner and outer surface; and,
an outer skin substantially covering the inner mechanism, wherein, the mechanism includes a means for expanding and contracting at least a portion of the housing to increase and decrease the surface area of the outer skin.

2. The sexual stimulation device of claim 1 further comprising at least one vibrating motor operatively attached to the inner mechanism.

3. The sexual stimulation device of claim 1 further comprising two or more separable panels.

4. The sexual stimulation device of claim 3, wherein the means for expanding and contracting the housing is positioned inside in the inner mechanism.

5. The sexual stimulation device of claim 3 further comprising a drive motor.

6. The sexual stimulation device of claim 1, wherein the means for expanding and contracting the housing includes a coiled spring.

7. The sexual stimulation device of claim 1, wherein the means for expanding and contracting the housing includes an angled surface operatively attached to the inner surface of the housing.

8. The sexual stimulation device of claim 7, wherein the means for expanding and contracting the housing further includes a shaft and a disc.

9. The sexual stimulation device of claim 8, wherein the means for expanding and contracting the housing further includes a planar surface extending longitudinally from the end of the shaft.

10. The sexual stimulation device of claim 9, wherein the means for expanding and contracting the housing further includes at least one guide pin extending substantially perpendicular from the planar surface.

11. The sexual stimulation device of claim 10, wherein the at least one guide pin operatively engages the angled surface to expand and contract the inner housing.

12. The sexual stimulation device of claim 1, wherein the outer skin includes raised portions.

13. The sexual stimulation device of claim 12, wherein the raised ridges are heated via infrared.

14. The sexual stimulation device of claim 3, wherein the outer skin covering at least one edge of the two or more separable panels includes a greater thickness than substantially the rest of the outer skin.

15. The sexual stimulation device of claim 3, wherein a flexible joint cover is located between the outer skin and an area between the two or more separable panels.

16. The sexual stimulation device of claim 1 wherein one side of the device includes a substantially perpendicular protrusion.

17. The sexual stimulation device of claim 16 wherein the substantially perpendicular protrusion is a vibrating motor surrounded by the outer skin.

18. The sexual stimulation device of claim 1 wherein the device is controlled by an external remote control.

19. An internal mechanism for a sexual stimulation device comprising:
a first housing;
a second housing having an inner and outer surface;
at least one angled surface extending from the inner surface of the second housing;
a planar surface extending inside the second housing substantially parallel to the at least one angled surface;
at least one guide pin extending substantially perpendicular from the planar surface, wherein the at least one guide pin operatively engages the at least one angled surface to expand and contract the second housing.

20. The internal mechanism of claim 19 further comprising a drive motor positioned within the first and/or second housing.

21. The internal mechanism of claim 20, further comprising a shaft operatively connected to the motor.

22. The internal mechanism of claim 21, wherein the planar surface is attached to the shaft.

23. The internal mechanism of claim 22, wherein the second housing expands and contracts as the motor laterally translates the shaft.

24. The internal mechanism of claim 19, further comprising at least one vibrating motor contained within the first and/or second housing.

25. The internal mechanism of claim 19, wherein the internal mechanism is controlled by an external remote control.
